(19) United States
(12) Patent Application Publication
(10) Pub. No.: US 2026/0215972 A1
(43) Pub. Date: Jul. 30, 2026

(54) Absorbent Assembly

(71) Applicant: Essity Hygiene and Health Aktiebolag, Göteborg (SE)

(72) Inventors: Anna KNÖS, Göteborg (SE); Philip BLOMSTRÖM, Göteborg (SE)

(73) Assignee: Essity Hygiene and Health Aktiebolag, Göteborg (SE)

(21) Appl. No.: 19/146,111

(22) PCT Filed: Feb. 9, 2023
(86) PCT No.: PCT/EP2023/053165

§ 371 Date: Jul. 7, 2025

(51) Int. Cl. A61F 13/49; A41B 9/12; A61F 13/511; A61F 13/515; A61F 13/537; A61F 13/539

(52) U.S. Cl. CPC A61F 13/49006; A41B 9/12; A61F 13/511; A61F 13/515; A61F 13/537; A61F 13/539; A61F 2013/53908; A61F 2013/53983

(57) ABSTRACT

A reusable absorbent assembly for use in an undergarment that is configured to be washable, having a shape with an extension in a longitudinal direction and in a transversal direction, the absorbent assembly including a wearer facing textile top layer, a liquid-impermeable barrier and an absorbent core assembly located in between the top layer and the liquid-impermeable barrier, said absorbent core assembly including a first absorbent layer and wherein the absorbent core assembly includes at least one rectangular shaped layer.

### FIELD OF THE INVENTION

The present disclosure relates to a reusable absorbent assembly for use in an undergarment that is configured to be washable, having a shape with an extension in a longitudinal direction and in a transversal direction, the reusable absorbent assembly comprising a wearer facing textile top layer, a liquid-impermeable barrier layer and an absorbent core assembly located in between the top layer and the liquid-impermeable barrier layer, said absorbent core assembly comprising a first absorbent layer.

### BACKGROUND ART

Such absorbent assemblies, generally known as an integrated part of washable and reusable absorbent undergarments and as washable and reusable absorbent pads, form an environmental and economical advantageous alternative to disposable sanitary napkins and disposable absorbent undergarments.

Washable and reusable absorbent undergarments conventionally comprise absorbent cores conventionally including woven, knit or cloth material. Such core materials have lower absorbent capacity compared to materials used for conventional absorbent cores in disposable absorbent articles, such as superabsorbent polymers and cellulosic fibers. The risk of leakage and rewetting may therefore be higher for reusable and washable absorbent undergarments. To increase the absorbency and thereby reducing the risk of leakage, such absorbent cores may comprise multiple core layers. However, such constructions may form a bulky structure being less comfortable compared to thinner disposable absorbent cores including superabsorbent polymers and/or cellulosic fibers.

Furthermore, a relatively high amount of material is used to manufacture washable and reusable absorbent cores to obtain the desired absorbency. Thus, from an environmental and economical perspective, savings in this area are desirable.

There is thus still a need for an absorbent core assembly which has improved comfort and fit, and which requires a relatively low amount of material for manufacturing, whilst at least maintaining leakage security.

### SUMMARY OF THE INVENTION

This object may be achieved with a reusable absorbent assembly for use in an undergarment in accordance with claim 1. Further embodiments are set out in the dependent claims and in the following description.

The present disclosure relates to a reusable absorbent assembly for use in an undergarment, having a shape with an extension in a longitudinal direction and in a transversal direction, the reusable absorbent assembly comprising a wearer facing textile top layer, a liquid-impermeable barrier and an absorbent core assembly located in between the top layer and the liquid-impermeable barrier, said absorbent core assembly comprising a first absorbent layer and wherein the absorbent core assembly comprises at least one rectangular shaped layer.

A rectangular shaped layer is a layer having an outer contour that is a four-sided shape, having two pairs of sides that are parallel to one another. The rectangular shaped layer can be easily cut multiple times from a main material source (often a large sheet or roll of fabric, film or laminate), with a single cutting line forming two contour sides of adjacent layers. Thus, the use of rectangular shaped layers reduces the amount of cutting and minimizes waste.

Furthermore, the cutting of layers with a rectangular contour from a main material source allows for multiple layers being cut directly adjacent to one another, without having to account for a positioning direction of each individual cut layer in the manufacturing line. Thus, no energy is wasted during production to account for variation in direction of each cut layer.

The reusable absorbent assembly is configured to be washable. Washable should be understood as being suitable to undergo at least 10 washing cycles at a temperature of at least 30° Celsius, without disintegrating. During the washing, the absorbent assembly releases the absorbed fluids, thereby regenerating the absorptive functioning, enabling the absorbent assembly to be reused for the same purpose again.

The shape and size of the reusable absorbent assembly may be such that at least a crotch section of said undergarment is covered during use. Thus, the entire crotch section is protected from soiling by the reusable absorbent assembly. A crotch section is a surface section having a size and shape to cover some or all of the genital area of a user, i.e. a surface section shaped to fit in between the legs of a user, that has a length sufficient to extend at least from the mons pubis to the perineum and a width that varies along the length. The crotch section thus has a width substantially corresponding to a transversal width in between leg openings of an undergarment.

At least 75% of the total length of side edges of the at least one rectangular shaped layer may be straight edges. Thus at least 75% of the outer edge of the at least one rectangular shaped layer conforms to the rectangular outer contour, thereby limiting the amount of material wasted, while allowing for some minimal shape variations in the layer that are required to assure wearer comfort and prevent the article from bulging during use.

At least 80% of a respective longitudinal length of each of the side edges of the at least one rectangular shaped layer may be straight. This allows for a relatively small cut-out of the layer being made to account for a reduced width between the legs. In particular, where multiple rectangular shaped layers are used, it may be beneficial to have at least one layer having a rectangular contour with a larger length and width than the further layers, to provide additional absorption capacity over a larger area to the absorbent core assembly. In this case, making a relatively small additional cut-out in the longitudinal side edges may allow the resulting absorbent core assembly to conform better to the crotch area of a wearer, making the assembly more comfortable and less visible when worn.

The side edges of the at least one rectangular shaped layer in the longitudinal direction and/or in the transversal direction may be entirely straight, such that said edges form the outer contour at the respective sides and no additional material is removed to form these edges. This allows for substantially the entire main fabric source being used as layers in the absorbent core assembly, without waste.

An angle between adjacent side edges of the at least one rectangular shaped layer may be between 85° and 90°, preferably 90°.

The top layer and the liquid-impermeable barrier layer may be substantially equal in size and shape and may have an outer contour that forms an outer contour of the reusable absorbent assembly. Through the liquid-impermeable barrier layer having the same size and shape as the top layer, the protection against leakage is available over the same area that is also directly visible to the user, giving a good indication to the user of the provided leakage protection by the absorbent assembly.

The absorbent core assembly may comprise a rectangular shaped first absorbent layer. An edge width of the first absorbent layer may be between 25%-100%, preferably between 25%-90%, of a front edge width of the reusable absorbent assembly and 80%-100% of a crotch width W_{C}, with all widths seen in the transversal direction x.

The absorbent core assembly may further comprise a moisture wicking layer, which is located between the absorbent layer and the top layer. This moisture wicking layer may be rectangular shaped. Furthermore, the moisture wicking layer may have a longitudinal length of at least 50 mm, said length being considered sufficient to cover a surface around the urethral and vaginal openings, which is where fluids are excreted that need wicking away.

An edge width of the moisture wicking layer may be 25%-100%, preferably 25%-90%, most preferred 30%-85% of a front edge width of the reusable absorbent assembly, with all widths seen in the transversal direction x.

The absorbent core assembly may further comprise a second absorbent layer, having a transverse edge width that is larger than a transverse edge width of the first absorbent layer. The shape of the second absorbent layer may be matching the shape of the top layer or may also be rectangular. By a matching shape is intended a shape that is substantially the same, although the size can differ and the edges of the shape may contain cut-outs, but only to an extend that outer ends of the shape can be interconnected to form an outline having a shape that is substantially the same. The larger transverse width ensures that a minimum absorbency is present over a larger area than provided by the first absorbent layer. Furthermore, the difference in width prevents the stacking of layer-edges, that could otherwise result in unwanted bulging of the absorbent assembly. The second absorbent layer may be located between the first absorbent layer and the top layer. An advantage with this order is a core assembly that more easily follows the shape of the body and thereby feels less bulky.

The absorbent core assembly may comprise at least two layers, wherein each of said at least two layers have an outer contour that terminate a different positions in the longitudinal direction and/or transversal direction on the extension of the reusable absorbent assembly. Thus, the absorbent core assembly may be non-symmetrical along any longitudinal and/or transversal axis. In this configuration, the edges of the layers are not stacked, thereby forming a smoother transition of layers over the surface of the reusable absorbent assembly and ensuring a more gradual change in properties. In particular, a gradual change in stiffness of the absorbent assembly is desirable, since this prevents the assembly from bulking up to form a sausage shape. Not only is this shape considered unflattering and therefore undesirable for female users, but the shape can also result in a gutter-shape occurring on the wearer-facing side, reducing direct contact between the assembly and the wearer and thereby reducing effective uptake of excreted liquids by the assembly.

The absorbent assembly may be substantially symmetrical along a central axis in the longitudinal direction, to aid correct placement of the assembly within the undergarment during manufacturing or, when in the form of a pad, during use.

A width of the absorbent core assembly, seen in transversal direction, may be less than 95% of a corresponding width of the reusable absorbent assembly. This allows for the barrier layer and top sheet being adhered to one another around the absorbent core assembly, effectively closing off said core assembly and preventing leakage at the edges.

All layers of the absorbent core assembly may be rectangular shaped, resulting in optimum waste reduction during the production of the core assembly. Furthermore, all layers of the reusable absorbent assembly may be rectangular shaped, to fully optimise use of resources during manufacturing.

To ensure the reusable absorbent assembly conforms to a wearer's lower body shape, over a predetermined length in the crotch section all layers of said reusable absorbent assembly may have a same maximum width in the transversal direction. Thus along the inner thighs the reusable absorbent assembly may be provided with leg seams, having a similar shape to the leg openings in undergarments, resulting in a comfortable fit in the crotch area. The required contouring for obtaining this local edge shape may be performed after assembling of the layers that form the reusable absorbent assembly. Thus, making a leg-shaped cut-out and joining of the contoured edges may be performed after all layers, including the at least one rectangular shaped layer, have been stacked.

As indicated above, the absorbent assembly may be configured as a pad that is adapted for detachably attaching to said undergarment. Thereto the absorbent assembly may comprise an adhesive layer or a hook patch.

Alternatively, the absorbent assembly may be configured as a gusset that is permanently attached to a wearer-facing side of a crotch section of a body fabric forming said undergarment. A gusset is an extra fabric layer(s) integrated into the crotch section of an undergarment, such as a panty or bikini brief, on a side of the crotch section that is facing the user's crotch during use. The gusset is thus permanently attached to a main body fabric of the undergarment. A smallest width of the reusable absorbent assembly may be equal to 80%-100%, preferably to 90%-100% of a smallest width of the crotch section X_{C} of the body fabric. This allows the rectangular layer to have longitudinal side edges that are fully straight, whilst being spaced sufficiently from the edges of the body fabric of the undergarment to allow joints/seams being located in between, without negatively impacting the shape or comfort of the crotch section. Depending on the particular layer, a smallest width of the at least one rectangular shaped layer, seen in transversal direction, may be equal to 35%-55% of a smallest width of the crotch section of the body fabric.

A method of manufacturing a reusable absorbent assembly for use in an undergarment, said reusable absorbent assembly having a shape with an extension in a longitudinal direction and in a transversal direction, may comprise the steps of:
providing a liquid permeable top layer, for facing a wearer during use, having a predetermined shape and size for covering at least a part of a crotch area
providing a liquid-impermeable barrier layer, having a shape and/or size matching the liquid permeable top layer, and
providing an absorbent core assembly, comprising at least one core layer and locating said absorbent core assembly in between the top layer and the liquid-impermeable barrier layer, and fastening edges of said top layer and barrier layer together,
   wherein the providing of the absorbent core assembly comprises the cutting of at least one rectangular shaped layer.

The method may further comprise a step of providing two leg-seams having contours corresponding to leg-openings in the undergarment, said leg-seams determining a maximum width in the transversal direction for all layers of the reusable absorbent assembly over a predetermined length in the crotch section. This may comprise cutting and stitching of at least some layers of the absorbent core assembly. Said cutting and stitching may be performed simultaneously and by a single machine. The rectangular layer may be attached e.g. by sewing to any other nearby layer before stitching and/or sealing the absorbent assembly.

### BRIEF DESCRIPTION OF THE DRAWINGS

Embodiments of a reusable absorbent assembly according to the present disclosure will be described by way of example, with reference to the attached drawings, in which:

FIG. 1 depicts a reusable absorbent assembly according to an embodiment of the present disclosure, in this case an undergarment with the reusable absorbent assembly permanently attached as a gusset,

FIG. 2 depicts another reusable absorbent assembly according to an embodiment of the present disclosure, in this case with the reusable absorbent assembly configured as a detachably attached pad in an undergarment,

FIG. 3 depicts a plan view of an inner side of the undergarment of FIG. 1, and

FIGS. 4, 5, 6 and 7 each show a cross-sectional view of a reusable absorbent assembly according to an embodiment of the present disclosure.

### DESCRIPTION OF EMBODIMENTS

As used herein, the term “reusable absorbent assembly” refers to assemblies that are worn and intended to be placed against the skin of a wearer to absorb and contain body fluids such as urine, and vaginal fluids including menstrual fluid. The reusable absorbent assembly may be intended for female or male use. The assembly according to the present disclosure is intended to be washable or otherwise restored after use for reuse as a sanitary article. The absorbent assembly may be used as and/or for a washable and reusable absorbent pad or napkin or be a part of an absorbent undergarment being underwear of a fabric or textile.

By “washable” it is meant that the absorbent undergarment may be cleaned by laundering. The absorbent undergarment may thus be subjected to an aqueous solution containing detergent without losing its structural features. This aqueous solution may be heated as part of the laundering process, e.g. to 30° C., 40° C. or 60° C. The laundering may be performed by a machine.

The term “textile” as used in the present disclosure may refer to single or multiple layers of fabrics that may be knitted or woven.

By “permanently attached”, it is meant that the absorbent assembly is not intended to be separated from the body fabric before, during, or after use. The absorbent assembly as proposed herein is not necessarily directly bonded to the fabric, but instead may be attached via the leg opening seams or bonded by other means such as sealing tape. The absorbent assembly per se as proposed herein may or may not be permanently attached to an undergarment fabric material.

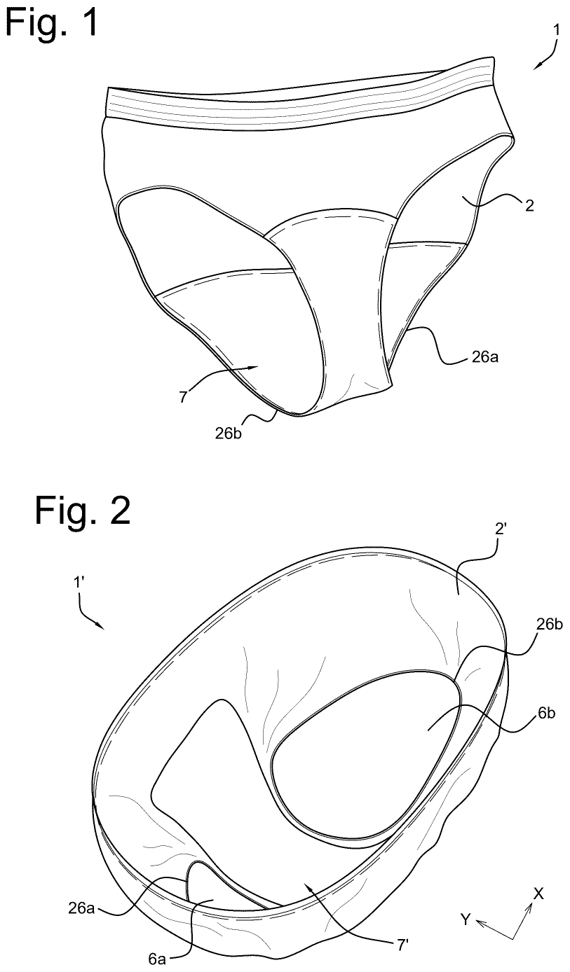

FIG. 1 depicts an undergarment 1 having a reusable absorbent assembly 7 according to an embodiment of the present disclosure permanently attached as a gusset. The absorbent undergarment 1 is comprises a main body material 2 and an integrated reusable absorbent assembly 7 that together are shaped to be worn around the lower part of a person's torso, and therefore are provided with a waist opening, defining an upper edge, and two leg openings where the main body material 2 is delimited by leg edges 26a, 26b.

The body material 2 may be constructed of any suitable textile fabric, such as conventional underwear or panty material. Suitable fabrics may include naturally derived fibers and mixtures thereof selected from the group consisting of cotton, wool, silk, cellulose, regenerated cellulose, rayon, viscose, modal, lyocell, bamboo, hemp, flax, ramie, coir or banana. Alternatively, the fabric may be constructed of synthetic fibers or mixtures thereof selected from the group consisting of polyamide, acrylic, polyester. Further, the fabric may be constructed of a blend or a mixture of naturally derived and/or synthetic fibers. The fibers may be recycled fibers. The fabric may comprise a stretchable fabric, e.g. elastane, so that the absorbent undergarment can provide a firm fit while at the same time adapting to the wearer's movements thus preventing any leakage from migrating through the leg openings and keeping the absorbent assembly in place. The fabric is preferably breathable to allow vapor to escape from the wearer's skin and from the absorbent structure.

The reusable absorbent assembly 7 is integrated in the undergarment as a gusset, a feature that is permanently attached to the main body material 2 to at least form a part of an inner surface of the undergarment in a crotch section thereof. The inner surface of the undergarment 1 is intended to be in direct contact with a wearer's skin during use. The layers of the reusable absorbent assembly 7 and the undergarment body fabric 2 may be bonded by conventional stitching, gluing techniques or via a bonding film such as tape.

FIG. 2 depicts another reusable absorbent assembly 7′ according to an embodiment of the present disclosure, in this case with the reusable absorbent assembly 7′ configured as a detachably attached pad in an undergarment 1′. Although the depicted undergarment 1′ is formed by a main body material 2′ that is shaped similarly to the undergarment depicted in FIG. 1, also having leg openings 6a, 6b delimited by leg edges 26a, 26b, and may be made of a similar body material, the undergarment itself is not configured to be absorbent like the undergarment of FIG. 1. Instead, the reusable absorbent assembly 7′, which may further be similarly shaped and configured to the gusset of FIG. 1, has been further modified to be detachably attachable to underwear, to cover a crotch section of an inner surface of said underwear.

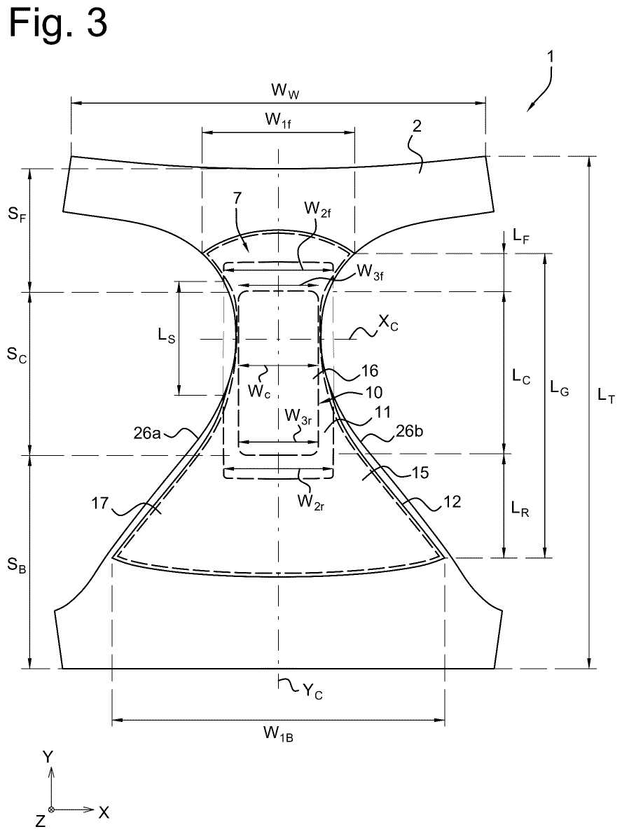

Further features of reusable absorbent assemblies according to the present disclosure are described in more detail with reference to FIGS. 3, 4, 5, 6 and 7, which respectively illustrate the undergarment of FIG. 1, in plan view and as seen from a user facing side, and some examples of layering options for the reusable absorbent assembly 7.

The undergarment 1 extends in the transverse direction X, having a maximum transversal width Ww at the front and back edges, chosen to fit around a person's waist, and a minimum crotch section width Wc, defining the smallest transversal width of the undergarment that is located in between the leg openings and defines a central transverse axis Xc. The undergarment 1 extends in the longitudinal direction, having a longitudinal length L_{T} that is subdivided into a front section S_{F}, a crotch section S_{C} and a back section S_{B}, the crotch section having a length L_{c}.

The reusable absorbent assembly 7 has an extension in the longitudinal direction Y and in the transverse direction X, said transverse direction X being perpendicular to the longitudinal direction Y. The absorbent assembly 7 further has a thickness direction Z, which is perpendicular to both the longitudinal and the transverse directions X, Y. The depicted undergarment 1 is symmetrical around a longitudinal central axis Y_{C}.

In the example of FIG. 3, the reusable absorbent assembly 7 has a length L_{G} that is longer than the length L_{c} of the crotch section S_{C}, such that the reusable absorbent assembly 7 extends over said crotch section and partially into the front section S_{F} and partially into the back section S_{B}. Depending on the particular absorptive design, i.e. the intended absorptive rating and intended use, such as for example during sleeping or sports, however, the length L_{G} of the reusable absorbent assembly may be shorter than the length L_{c} of the crotch section and may only cover a predetermined part of said crotch section S_{c} and optionally a predetermined length of the back section S_{B}. The length of the reusable absorbent assembly L_{G} may be between 50 and 500 mm. The length L_{G} may be symmetrically distributed around the central transverse axis X_{C} or may have a greater extension towards the back section than towards the front section with respect to said central transverse axis X_{C}. Any layer of the core assembly may be between 10 and 100% of the length of the reusable absorbent assembly L_{G}.

The reusable absorbent assembly 7 comprises a wearer facing textile top layer 15, a liquid-impermeable barrier layer 12 and an absorbent core assembly 10 located in between the top layer and the liquid-impermeable barrier layer in the thickness direction Z. During use, the top layer 15 forms an inner side, contacting the wearer's skin.

The reusable absorbent assembly 7 may comprise woven material, a nonwoven and/or a knit material. The fibers in the absorbent assembly material may be synthetic fiber, such as polyamid, polyester and polyolefin fibers, and/or wool fibers, and the other one of the first or the second absorbent layer may comprise cotton fibers, rayon fibers, modal fibers, bamboo fibers, merino wool fibers and/or recycled fiber. Herein, recycled fibers originate from reclaimed pre- or post-consumer waste materials that are converted into new fibers through a textile recycling process to create upcycled new textiles.

The wearer facing textile top layer 15 may have a basis weight of from 80 gsm or from 120 gsm. The wearer facing textile top layer 15 may have a basis weight of up to 350 gsm. The wearer facing textile top layer 15 may be a woven material, and/or a knit material layer(s).

The liquid-impermeable barrier 12 prevents liquid from leaking through the absorbent core into the main body material 2 of the undergarment. The liquid-impermeable barrier 12 may be a coating on the wearer facing side of the fabric layer 2 or on a garment facing side of the absorbent layer 11. The barrier layer 12 may comprise a plastic film, the plastic film may be a breathable plastic film. The barrier layer 12 may comprise any suitable material or combinations of material that prevents liquid from migrating from the absorbent assembly to the fabric layer. The liquid-impermeable barrier layer 12 may comprise a hydrophobic woven or nonwoven material having inherent hydrophobic properties, or that has been treated to become hydrophobic. Examples of hydrophobic materials for treating the barrier layer are polymers such as silicone, polyurethane and combinations thereof. The liquid-impermeable barrier layer 12 may comprise a microporous polymer film, e.g. a polyethylene, PTFE or polyurethane film, or combinations thereof. Laminates of polymer films and nonwoven materials may also be used. The liquid-impermeable barrier layer 12 may be a coating of a moisture-impermeable material. The coating may be a polymer such as urethane wax on the surface facing away from the wearer. The liquid-impermeable barrier layer 12 may be of polyurethane. The liquid-impermeable barrier layer 12 is preferably breathable, to allow vapor to escape from the absorbent undergarment, while preventing liquid from passing through the fabric layer. Further, the liquid-impermeable barrier layer 12 may be constructed of an elastic material, thus providing the absorbent assembly with the required flexibility to adapt to the user's anatomy and movements. This improved fit increases the wearer's comfort during use and helps to prevent leakage from migrating through the undergarment's leg openings 6a, 6b.

The absorbent core assembly 10 has at least one core layer for absorbing liquids, and has at least one layer with a rectangular shaped outline. The four sides of the rectangular shaped outline form two side pairs that are parallel to one another. In the example depicted in FIG. 3, the absorbent core 10 has a rectangular shaped moisture wicking layer 11, a rectangular shaped absorbent core layer 16 and a further absorbent core layer 17. A length of this rectangular shaped core layer 16 may be substantially corresponding to a length of the L_{C} of the crotch section, this length being shorter than the length L_{G} of the reusable absorbent assembly, with a distance between the front edge of the rectangular shaped absorbent core layer 16 and the front edge of the reusable absorbent assembly being L_{F} and a distance between the back edge of the rectangular shaped absorbent core layer 16 and the back edge of the reusable absorbent assembly being L_{R}. Thus, the rectangular absorbent core layer 16 provides fluid retention capacity in the crotch section, where the largest amount of fluid usually is excreted. The four sides of the rectangular shaped moisture wicking layer 11 and of the rectangular shaped absorbent core 16 are at 90° angles to one another, however, smaller or larger angles could be used without losing the benefit of reduced waste during layer cutting.

The further absorbent layer 17 is shaped to have an outline that matches the outline of the top layer 15 and the barrier layer 12, which in the depicted example conform to the local shape of the undergarment in which the reusable absorbent assembly 7 is located and are therefore non-rectangular, with a front edge width W_{1F} being smaller than a back edge width W_{1B} and both the front and back edge widths W_{1F}, W_{1B} being larger than a minimum crotch section width W_{C}. Thus the further absorbent layer 17 extends beyond the rectangular shaped core layer, providing additional fluid storage capacity to the reusable absorbent assembly. The particular outline shape of the top layer 15 and barrier layer 12, however, may be different from the depicted shape, resulting in an overall narrower shape that may, for example, also be substantially rectangular.

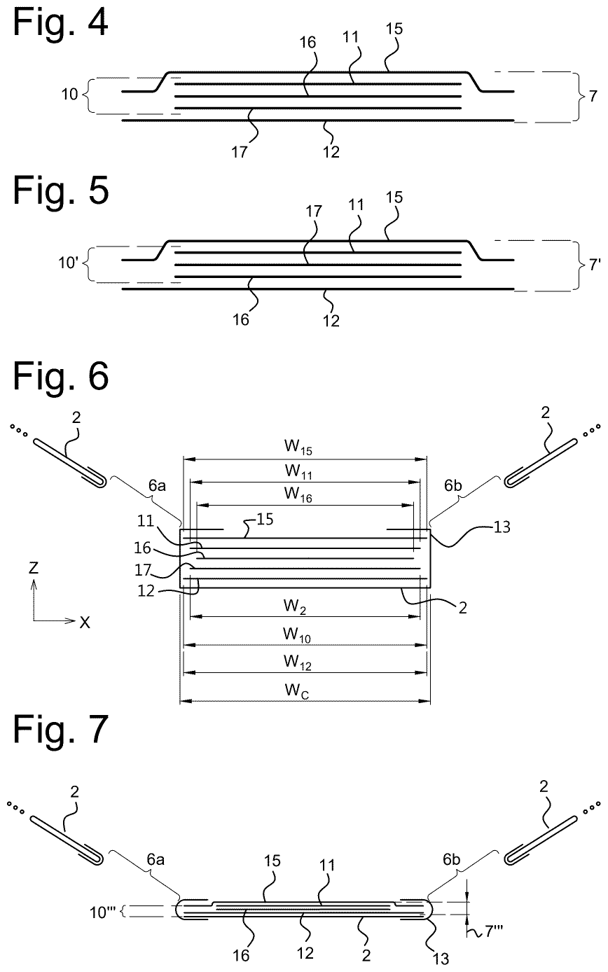

FIG. 4 depicts a cross-sectional view of the reusable absorbent assembly 7 shown in FIG. 3 at the central transverse axis X_{C}, showing the rectangular core layer 16 located in between the moisture wicking layer 11 and the further absorbent core layer 17. Alternatively, the further absorbent core layer 17 may be located in between the moisture wicking layer 11 and the rectangular shaped core layer 16 in the reusable absorbent assembly 10′, as depicted in FIG. 5.

Since the reusable absorbent assembly has at least one core layer for absorbing liquids and has at least one layer with a rectangular shaped outline, in FIG. 3, the depicted moisture wicking layer 11 or the rectangular shaped absorbent core layer 16 may thus be considered an optional layer. Alternatively or additionally, the further absorbent core layer 17 may be considered an optional layer. The absorbent core assembly 10′″ may consist of a moisture wicking layer 11 and an absorbent layer 16 as depicted in FIG. 7, with at least one of the layers having a rectangular shaped outline. Additional absorbent core layers may be included, which may also have a rectangular shaped outline.

All layers of the absorbent core assembly, which may include a rectangular moisture wicking layer 11, and/or a rectangular shaped core layer 16 and an optional further core layer 17, overlap each other in the crotch section Sc of the absorbent core assembly 10, as depicted in FIG. 3, providing an improved absorbency in the overlap section. The area of all layers overlapping coincides with a part of the crotch section Sc which, during use, is in direct contact with the wearers body parts producing fluids. The rectangular shaped absorbent layer 16 has a front edge width W_{3F} that is equal to a back edge width W₃ᵣ. Similarly, the depicted optional rectangular shaped wicking layer 11 has a front edge width W_{2F} that is equal to a back edge width W₂ᵣ. In the depicted example, the front width W_{3F} and back width W₃ᵣ of the first absorbent layer 16 correspond substantially to the minimum crotch sectional width W_{C}.

To make the reusable absorbent assembly 7 comfortable and discrete to wear, preventing unwanted bulging, while providing leakage protection over a substantial area, the reusable absorbent assembly 7 may have a reducing thickness towards the edges, achieved through fewer layers being present. Hereto, the (various) layer(s) of the absorbent core assembly 10 may terminate at different positions from further layers of the reusable absorbent assembly, including the top layer and the barrier layer. As in the depicted example of FIG. 3, the front and back edge widths of the subsequent layer, in this case the widths of the rectangular shaped layer W_{2F}, W₂ᵣ, of the wicking layer 11 are larger than the front and back edge widths W_{3F}, W₃ᵣ of the first absorbent layer 16.

In order to comfortably fit in between the legs, each width of the reusable absorbent assembly in transversal direction x along the longitudinal axis y may be maximized to a respective corresponding crotch section width, such that a width of the reusable absorbent assembly at the central transverse axis is equal or less than the minimum crotch width W_{C}. Hereto, over a predetermined longitudinal length L_{S}, the reusable absorbent assembly 7 may be provided with two leg-seams, having contours substantially corresponding to leg-openings 26a, 26b in the undergarment as shown in FIG. 3. In order to conform to the shape of the crotch section Sc, one (as depicted in FIG. 3) or more of the rectangular shaped layers, when having a front and rear edge width larger than the minimum crotch section width W_{c}, may have a section cut out from a longitudinal side edge. A total area of cut-out sections may represent less than 15% of a total area of a respective rectangular shaped layer.

Generally speaking, a cross-section of the reusable absorbent assembly taken in transversal direction x may look like one of FIG. 4, 5, 6 or 7, where a width of a respective absorbent layer and/or moisture wicking layer may be up to 100% of the width of the top layer 15 and/or barrier layer 12. Having a small difference in width between the absorbent layer and/or moisture wicking layer and the barrier layer 12 allows for the making of leak-proof seams, encapsulating the absorbent core assembly. Referring to FIG. 6, where widths are indicated for various layers in the undergarment, the maximum width W₁₀ for layers of the absorbent core assembly 10″ may be between 80% and 95% of the width of the barrier layer W₁₂. The width of the barrier layer W₁₂ may be equal to the width W₂ of the body material 2, in particular when the reusable absorbent assembly 7″ is integrated in the undergarment as in the examples of FIGS. 3 and 6.

The leg edges 26a, 26b of the main body material 2 and/or the reusable absorbent assembly 7 may be provided with a leg opening seam 13, which may comprise an elastic member such as elastic bonding film of polyurethane. Further, this leg opening seam 13 may serve to bond the reusable absorbent assembly 7, such as the top layer 15, the outer liquid impermeable barrier layer 12 and all or part of the absorbent core assembly 10 together, as depicted in FIGS. 6 and 7. Alternatively, the main body material 2 may be folded around the edges of the absorbent assembly and/or bonded to the reusable absorbent assembly 7. The leg openings and the absorbent assembly may be sealed with a tape (not shown) to avoid side leakage at the leg openings.

Where a section is cut out from one or more layers of the absorbent core, as described above for FIG. 3, a width of the rectangular shaped wicking layer 11 W₁₁, at the transversal central axis X_{C}. may be substantially equal to any further layers of the absorbent core assembly 10″, and may be substantially equal to a width W₁₂ of the barrier layer. This may be achieved through all layers being cut and stitched together, for forming the leg seams, simultaneously by a single machine. In particular when the reusable absorbent assembly 7″ is an integrated part of an undergarment, this cutting and stitching may be performed simultaneously with the inclusion of leg opening seams 13 at the leg edges 26a, 26b of the undergarment, wherein the width W₁₂ of the barrier layer substantially corresponds to the width W₂ of the body material, both corresponding substantially to the minimum crotch section width W_{C}, at the transversal central axis X_{C}.

The present invention has been described with reference to a number of explanatory embodiments as shown in the drawings. Obvious modifications and alternative implementations of some parts or elements will be apparent to those skilled in the art upon reading and understanding the foregoing detailed description. In particular, it will be obvious to the skilled person that various alternatives explicitly mentioned in the detailed description may be implemented independently from other explicitly mentioned alternatives that are not explicitly described as being related thereto. The invention is intended to be construed as encompassing all such modifications and alternative implementations to the extent that they fall within the scope of the appended claims.

1. A reusable absorbent assembly for an undergarment that is configured to be washable, having a shape with an extension in a longitudinal direction and in a transversal direction, the reusable absorbent assembly comprising a wearer facing textile top layer, a liquid-impermeable barrier and an absorbent core assembly located in between the top layer and the liquid-impermeable barrier, said absorbent core assembly comprising a first absorbent layer and wherein the absorbent core assembly further comprises a second absorbent layer, having a transverse edge width that is larger than a transverse edge width of the first absorbent layer and the absorbent core assembly comprises at least one rectangular shaped layer.

2. The reusable absorbent assembly, according to claim 1, wherein the shape and size of said reusable absorbent assembly is such that at least a crotch section of said undergarment is covered during use.

3. The reusable absorbent assembly according to claim 1, wherein at least 75% of the total length of side edges of the at least one rectangular shaped layer are straight edges.

4. The reusable absorbent assembly according to claim 1, wherein at least 80% of a respective longitudinal length of each of the side edges of the at least one rectangular shaped layer is straight.

5. The reusable absorbent assembly according to claim 1, wherein the side edges of the at least one rectangular shaped layer in the longitudinal direction and/or in the transversal direction are straight over the whole length.

6. The reusable absorbent assembly according to claim 1, wherein an angle between adjacent side edges of the at least one rectangular shaped layer is between 85° and 95°.

7. The reusable absorbent assembly according to claim 1, wherein the top layer and the liquid-impermeable barrier layer are substantially equal in size and shape and have an outer contour that forms an outer contour of the reusable absorbent assembly.

8. The reusable absorbent assembly according to claim 1, wherein the first absorbent layer is rectangular shaped.

9. The reusable absorbent assembly according to claim 8, wherein an edge width of the first absorbent layer is between 25%-100% of a front edge width of the reusable absorbent assembly and 80%-100% of a crotch width, with all widths seen in the transversal direction.

10. The reusable absorbent assembly according to claim 1, wherein the absorbent core assembly further comprises a moisture wicking layer, which is located between the absorbent layer and the top layer.

11. The reusable absorbent assembly according to claim 10, wherein the moisture wicking layer is rectangular shaped.

12. The reusable absorbent assembly according to claim 10, wherein an edge width of the moisture wicking layer is between 25%-100% of a front edge width of the reusable absorbent assembly, with all widths seen in the transversal direction.

13. (canceled)

14. The reusable absorbent assembly according to claim 1, wherein the second absorbent layer is located between the first absorbent layer and the top layer.

15. The reusable absorbent assembly according to claim 1, wherein the absorbent assembly comprises at least two layers, wherein each of said at least two layers have an outer contour that terminate at different positions in the longitudinal direction and/or transversal direction on the extension of the reusable absorbent assembly.

16. The reusable absorbent assembly according to claim 1, wherein the absorbent assembly is substantially symmetrical along a central axis in the longitudinal direction.

17. The reusable absorbent assembly according to claim 1, wherein all layers of the absorbent core assembly are rectangular shaped.

18. The reusable absorbent assembly according to claim 1, wherein all layers of the reusable absorbent assembly according to claim 1 are rectangular shaped.

19. The reusable absorbent assembly according to claim 1, wherein over a predetermined length in the crotch section all layers of said reusable absorbent assembly have a same maximum width in the transversal direction.

20. The reusable absorbent assembly according to claim 1, wherein the absorbent assembly is configured as a pad that is adapted for detachably attaching to said undergarment.

21. The reusable absorbent assembly according to claim 1, wherein the absorbent assembly is permanently attached to a wearer-facing side of a crotch section of a body fabric forming said undergarment.

22. The reusable absorbent assembly according to claim 21, wherein a smallest width of the reusable absorbent assembly is equal to 80%-100% of a smallest width of the crotch section of the body fabric.
